(19) United States
(12) Patent Application Publication
(10) Pub. No.: US 2007/0231384 A1
(43) Pub. Date: Oct. 4, 2007

(54) METHODS OF PROVIDING CONTROLLED-RELEASE PHARMACEUTICAL COMPOSITIONS AND CONTROLLED-RELEASE PHARMACEUTICAL COMPOSITIONS

(76) Inventors: Garratt W. Ponder, Wilmington, NC (US); Thomas W. Leonard, Wilmington, NC (US); Christopher L. Holshouser, Wilmington, NC (US)

Correspondence Address: MYERS BIGEL SIBLEY & SAJOVEC, PO BOX 37428, RALEIGH, NC, 27627, US

(21) Appl. No.: 11/762,313

(22) Filed: Jun. 13, 2007

Related U.S. Application Data
(63) Continuation of application No. 10/364,259, filed on Feb. 11, 2003.
(60) Provisional application No. 60/427,442, filed on Nov. 19, 2002.

(51) Int. Cl. A61K 9/22

(52) U.S. Cl. 424/468

(57) ABSTRACT

Methods are disclosed for forming a solid pharmaceutical composition having a desired release profile that include selecting hydroxypropylmethyl cellulose having a particular particle distribution to obtain the desired release profile, and forming a solid pharmaceutical composition comprising a core that comprises a bio-active and the hydroxypropylmethyl cellulose. Solid pharmaceutical compositions are also disclosed that include a bio-active, and hydroxypropylmethyl cellulose having a particle size that is selected to obtain a desired release profile.

### CROSS REFERENCE TO RELATED APPLICATIONS

The instant application is a continuation application of U.S. application Ser. No. 10/364,259, filed Feb. 11, 2003 and claims priority from U.S. Provisional Patent Application 60/427,442, filed Nov. 19, 2002, the disclosures of which are incorporated herein by reference in their entireties.

### FIELD OF THE INVENTION

This invention relates to pharmaceutical compositions, more particularly to pharmaceutical compositions including hydroxypropylmethyl cellulose.

### BACKGROUND OF THE INVENTION

WO 99/39698 proposes a sustained release tablet formulated containing a pharmaceutical and a three component release rate controlling matrix composition. The three components of the matrix composition are (1) a water insoluble polymer, such as ethyl cellulose, (2) a pH dependent gelling polymer, such as sodium alginate, and (3) a pH dependent gelling polymer, such as hydroxypropylmethyl cellulose.

WO 98/53803 proposes an enterie coated pharmaceutical formulation having a core material of the active ingredient omeprazole, an enteric coating, and a separating layer between the enteric coating and the active ingredient. The separating layer includes a specific quality of low viscosity hydroxypropylmethyl cellulose (HPMC). The HPMC preferably has a viscosity less than 7.2 cps in 2% aqueous solution and a cloud point of at least 45.6° C. determined by a Mettler instrument.

WO 98/47491 proposes an extended release dosage composition of pharmaceutically active substances that have a water contact angle (0) such that cos θ is between +0.9848 and −0.9848 presented in a matrix tablet. The pharmaceutically active substance is in intimate mixture with a polymer blend including, for example, ethylcellulose and hydroxypropylmethyl cellulose. The release of the pharmaceutically active substance is provided due to the unique mixture of the rate controlling constituents and excipients in selected ratios.

### SUMMARY OF THE INVENTION

The present invention relates to methods of providing controlled-release pharmaceutical compositions comprising hydroxypropylmethyl cellulose (HPMC). The invention further relates to controlled release pharmaceutical compositions comprising HPMC. While the references discussed above may provide controlled release pharmaceutical compositions that contain HPMC, the compositions proposed by these references derive their controlled-release characteristics by, for example, providing a particular blend of polymers including HPMC, or selecting HPMC having a particular viscosity. The inventors have unexpectedly discovered that the release characteristics of a pharmaceutical composition can be controlled by selecting HPMC having a particular particle size distribution. The inventors have further discovered that the release characteristics can be controlled by including the HPMC having a selected particle size distribution in the core of the pharmaceutical composition.

According to embodiments of the present invention, a method of forming a solid pharmaceutical composition having a desired release characteristic is provided. The method includes selecting hydroxypropylmethyl cellulose having a particular particle distribution to obtain the desired release characteristic, and forming a solid pharmaceutical composition including a bio-active and the hydroxypropylmethyl cellulose.

According to other embodiments of the present invention, a method of forming a solid pharmaceutical composition having a desired release profile is provided. The method includes selecting hydroxypropylmethyl cellulose having a particular particle distribution to obtain the desired release profile, and forming a solid pharmaceutical composition including a bio-active and the hydroxypropylmethyl cellulose.

According to still other embodiments of the present invention, a pharmaceutical composition includes a bio-active and hydroxypropylmethyl cellulose having a particle size that is selected to obtain a desired release characteristic.

### DETAILED DESCRIPTION OF PREFERRED EMBODIMENTS

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “controlled release” is intended to mean the release of a bio-active at a pre-selected or desired rate. This rate will vary depending upon the application. Desirable rates include fast or immediate release profiles as well as delayed, sustained or sequential release profiles. Combinations of release patterns, such as initial spiked release followed by lower levels of sustained release of the bio-active are also contemplated by the present invention.

As used herein, the term “bio-active” includes therapeutic agents such as pharmaceutical or pharmacological active agents, e.g., drugs and medicaments, as well as prophylactic agents, diagnostic agents and other chemicals or materials useful in treating or preventing conditions, infections and/or diseases found in animals. The compositions of the present invention are particularly effective in humans and other mammals, but are intended for use in other animals such as fish and birds, or plants, insects and other organisms.

As used herein, the term “10% cumulative weight percentage” means that 10 weight percent of particles in a particle size distribution are less than the indicated size or within the indicated size range.

As used herein, the term “50% cumulative weight percentage” means that 50 weight percent of particles in a particle size distribution are less than the indicated size or within the indicated size range.

As used herein, the term “90% cumulative weight percentage” means that 90 weight percent of particles in a particle size distribution are less than the indicated size or within the indicated size range.

According to embodiments of the present invention, a method of forming a solid pharmaceutical composition having a desired controlled-release profile is provided. The method includes selecting hydroxypropylmethyl cellulose (HPMC) having a particular particle size distribution to obtain the desired controlled-release profile, and forming a solid pharmaceutical composition including a bio-active and the HPMC.

Preferably, the pharmaceutical composition contains between a lower limit of about 5, 10, 20, 30 or 40 and an upper limit of about 60, 70, 80, 90 or 95 percent by weight HPMC. More preferably, the pharmaceutical composition contains between a lower limit of about 5, 10, 15, 25 or 30 and an upper limit of about 50, 55, 60, 65 or 70 percent by weight HPMC. The pharmaceutical composition contains between a lower limit of about 1, 5, 10, 20 or 30 and an upper limit of about 70, 80, 90 or 95 percent bio-active. The pharmaceutical composition preferably contains between a lower limit of about 1, 5, 10, 15 or 20 and an upper limit of about 30, 35, 40, 45 or 50 percent by weight bio-active, and, more preferably, contains between a lower limit of about 5, 7, 10 or 12 and an upper limit of about 15, 17, 20, 22 or 25 percent by weight bio-active.

In preferred embodiments, the HPMC and the bio-active are blended together to form an HPMC/bio-active mixture. The HPMC/bio-active mixture is preferably homogeneous. The HPMC/bio-active mixture may be used in various ways within the solid pharmaceutical composition. For example, when the solid pharmaceutical composition is a multi-layer tablet having a core and one or more layers, the core of the tablet may include the HPMC/bio-active mixture and/or one or more of the layers of the tablet may include the HPMC/bio-active mixture. When the solid pharmaceutical composition is a tablet, the core preferably comprises the HPMC/bio-active mixture, and, more preferably, the core consists essentially of the HPMC/bio-active mixture. The ratio of HPMC: bio-active in the HPMC/bio-active mixture is preferably selected based on various factors including, but not limited to, the potency of the compound and the hydrophobic nature of the bio-active. For example, for low potency bio-actives, a sufficient amount of bio-active is needed to achieve sustained release. As another example, as the hydrophobicity of the bio-active increases, less HPMC may be required.

The particular particle size distribution of the HPMC selected to obtain a desired controlled-release profile may vary depending upon the bio-active to be include in the pharmaceutical composition. For example, a first pharmaceutical composition including a first bio-active and HPMC having a particular particle size distribution may have a quick release profile while a second pharmaceutical composition including a second bio-active and HPMC having the same particular particle size distribution as the HPMC included in the first pharmaceutical composition may have a sustained-release profile.

Those skilled in the art will be able to select HPMC having an appropriate particle size distribution to obtain a desired release characteristic for a given bio-active without undue experimentation. For example, one skilled in the art can determine the HPMC particle size distribution that is needed by forming a pharmaceutical composition including HPMC of a particular particle size distribution and a bio-active, for example, as described in the Examples below. The release profile of the pharmaceutical composition can then be determined, for example, as described in the Examples below. If the experimentally determined release profile is not the desired release profile, HPMC having a different particle size distribution may be selected and the steps of forming a pharmaceutical composition and determining the release profile of the composition may be repeated. The selecting, forming, and determining steps may be repeated until the experimental release profile approximates the desired release profile. In general, as the HPMC particle size is decreased, the release profile may tend to move from quicker release to more sustained release.

The bio-active may be selected from various bio-actives that can be formulated in a solid composition for oral delivery. Representative non-limiting classes of bio-actives useful in embodiments of the present invention include those falling into the following therapeutic categories: ace-inhibitors; anti-anginal drugs; anti-arrhythmias; anti-asthmatics; anti-cholesterolemics; anti-convulsants; anti-depressants; anti-diarrhea preparations; anti-histamines; anti-hypertensive drugs; anti-infectives; anti-inflammatory agents; anti-lipid agents; anti-manics; anti-nauseants; anti-stroke agents; anti-thyroid preparations; anti-tumor drugs; anti-tussives; anti-uricemic drugs; anti-viral agents; acne drugs; alkaloids; amino acid preparations; anabolic drugs; analgesics; anesthetics; angiogenesis inhibitors; antacids; antiarthritics; antibiotics; anticoagulants; antiemetics; antiobesity drugs; antiparasitics: antipsychotics; antipyretics; antispasmodics; antithrombotic drugs; anxiolytic agents; appetite stimulants; appetite suppressants; beta blocking agents; bronchodilators; cardiovascular agents; cerebral dilators; chelating agents; cholecystokinin antagonists; chemotherapeutic agents; cognition activators; contraceptives; coronary dilators; cough suppressants; decongestants; deodorants; dermatological agents; diabetes agents; diuretics; emollients; enzymes; erythropoietic drugs; expectorants; fertility agents; fungicides; gastro-intestinal agents; growth regulators; hormone replacement agents; hyperglycemic agents; hypnotics; hypoglycemic agents; laxatives; migrain treatments; mineral supplements; mucolytics; narcotics; neuroleptics; neuromuscular drugs; NSAIDS; nutritional additives; peripheral vaso-dilators; polypeptides; prostaglandins; psychotropics; renin inhibitors; respiratory stimulants; steroids; stimulants; sympatholytics; thyroid preparations; tranquilizers; uterine relaxants; vaginal preparations; vaso-constrictors; vaso-dilators; vertigo agents; vitamins; wound healing agents.

Examples of specific bio-actives which may be useful in embodiments of the present invention include, but are not limited to: acetaminophen; acetic acid; acetylsalicylic acid and its buffered form; albuterol and its sulfate; alcohol; alkaline phosphatase; allantoin; aloe; aluminum acetate, carbonate, chlorohydrate, hydroxide; alprozolam; amino acids; aminobenzoic acid; amoxicillin; ampicillin; amsacrine; amsalog; androgens; anethole; ascorbic acid; aspartame; atenolol; bacitracin; balsam peru; BCNU (carmustine) beclomethasone dipropionate; benzocaine; benzoic acid; benzophenones; benzoyl peroxide; bethanechol; biotin; bisacodyl; bornyl acetate; bromopheniramine maleate; buspirone; caffeine; calamine; calcium, calcium carbonate, casinate and hydroxide; camphor; captopril; cascara sagrada; castor oil; cefaclor; cefadroxil; cephalexin; cetylalcohol; cetylpyridinium chloride; chelated minerals; chloramphenicol; chlorcyclizine hydrochloride; chlorhexidine gluconate; chloroxylenol; chloropentostatin; chlorpheniramine maleate; cholestyramine resin; choline bitartrate; chondrogenic stimulating protein; cimetidine hydrochloride; cinnamedrine hydrochloride; citalopram; citric acid; cocoa butter; cod liver oil; codeine and codeine phosphate; clonidine and its hydrochloride salt; clorfibrate; cortisone acetate; ciprofloxacin HCl; cyanocobalamin; cyclizine hydrochloride; danthron; dexbrompheniranime maleate; dextromethorphan hydrobromide; diazapam; dibucaine; diclofenac sodium; digoxin; diltiazem; dimethicone; dioxybenzone; diphenhydramine citrate; diphenhydramine hydrochloride; docusate calicum, potassium and sodium; doxycycline hyclate; doxylamine succinate; efaroxan; enalapril; enoxacin; erythromycin; estrogens; estropipate; ethinyl estradiol; ephedrine; epinephrine bitartrate; erythropoietin; eucalyptol; ferrous fumarate, gluconate and sulfate; folic acid; fosphenytoin; 5-fluorouracil (5-FU) fluoxetine HCl; furosemide; gabapentan; gentamicin; gemfibrozil; glipizide; glycerin; glyceryl stearate; griseofulvin; growth hormone; guaifenesin; hexylresorcinol; hydrochlorothiaxide; hydrocodone bitartrate; hydrocortisone and its acetate; 8-hydroxyquinoline sulfate; ibuprofen; indomethacin; inositol; insulin; iodine; ipecac; iron; isoxicam; ketamine; koalin; lactic acid; lanolin; lecithin; leuprolide acetate; lidocaine and its hydrochloride salt; lifinopril; liotrix; lovastatin; luteinizing hormone; LHRH (luteinizing hormone releasing hormone); magnesium carbonate, hydroxide, salicylate, trisilocate; mefenamic acid; meclofenanic acid; meclofenamate sodium; medroxyprogesterone acetate; methenamine mandelate; menthol; meperidine hydrochloride; metaproterenol sulfate; methyl nicotinate; methyl salicylate; methylcellulose; methsuximide; metronidazole and its hydrochloride; metoprolol tartrate; miconazole nitrate; mineral oil; minoxidil; morphine; naproxen and its sodium salt; nifedipine; neomycin sulfate; niacin; niacinamide; nicotine; nicotinamide; nitroglycerin; nonoxynol-9; norethindone and its acetate; nystatin; octoxynol; octoxynol 9; octyl dimethyl PABA; octyl methoxycinnamate; omega-3 polyunsaturated fatty acids; omeprazole; oxolinic acid; oxybenzone; oxtriphylline; para-aminobenzoic acid (PABA); padimate 0; paramethadione; pentastatin; peppermint oil; pentaerythriol tetranitrate; pentobarbital sodium; pheniramine maleate; phenobarbital; phenol; phenolphthalein; phenylephrine hydrochloride; phenylpropanolamine and its hydrochloride salt; phenytoin; phenelzine sulfate; pirmenol; piroxicam; polymycin B sulfate; potassium chloride and nitrate; prazepam; procainamide hydrochloride; procaterol; propoxyphene and its HCl salt; propoxyphene napsylate; pramiracetin; pramoxine and its hydrochloride salt; propronolol HCl; pseudoephedrine hydrochloride and sulfate; pyridoxine; quinapril; quinidine gluconate and sulfate; quinestrol; ralitoline; ranitadine; resorcinol; riboflavin; salicylic acid; sesame oil; shark liver oil; simethicone; sodium bicarbonate, citrate and fluoride; sodium monofluorophosphate; sucralfate; sulfanethoxazole; sulfasalazine; sulfur; tacrine and its HCl salt; theophylline; terfenidine; thioperidone; trimetrexate; triazolam; timolol maleate; tretinoin; tetracycline hydrochloride; tolmetin; tolnaftate; triclosan; triprolidine hydrochloride; undecylenic acid; vancomycin; verapamil HCl; vidaribine phosphate; vitamins A, B, C, D, B₁, B₂, B₆, B₁₂, E, and K; witch hazel; xylometazoline hydrochloride; zinc; zinc sulfate; and zinc undecylenate. Mixtures of these agents and their esters or pharmaceutically acceptable salts, solvates, hydrates, and/or polymorphs used for appropriate therapies are also contemplated.

Preferably, the bio-active comprises a hormonal compound such as an estrogenic compound, an androgenic compound, a progestin, or mixtures thereof. More preferably, the bio-active comprises an estrogenic compound. In some embodiments, the bio-active may also comprise an additional active ingredient such as calcium salts, vitamin D, or a vitamin D derivative (e.g., cholecalciferol (Vitamin D₂), ergocalciferol (Vitamin D₃), and dihydrotachysterol as described in GOODMAN & GILMAN'S, THE PHARMACOLOGICAL BASIS OF THERAPEUTICS 1529-1536 (9^{th} ed. 1996) as well as provitamins and previtamins that are converted in the body to such substituted compounds).

Estrogenic compounds may be present in various forms, including, but not limited to, estrogenic ketones and their corresponding 17α- and 17β-hydroxy derivatives. For example, the estrogenic compounds may include estrone, 17α-estradiol, 17β-estradiol, equilin, 17α-dihydroequilin, 17β-dihydroequilin, equilenin, 17β-dihydroequilenin, 17β-dihydroequilenin, Δ^{8,9}-dehydroestrone, 17βΔ^{8,9}-dehydroestradiol, 17βΔ^{8,9}-dehydroestradiol, 6-OH equilenin, 6-OH 17α-dihydroequilenin, and 6-OH 17β-dihydroequilenin. The estrogenic compounds may also be present as conjugated estrogens. The conjugates may be various conjugates understood by those skilled in the art, including, but not limited to, glucuronide and sulfate. The most preferred conjugate is sulfate. The estrogenic compounds may also be present as salts of conjugated estrogens. The salts may be various salts understood by those skilled in the art, including, but not limited to, sodium salts, calcium salts, magnesium salts, lithium salts, and amine salts such as piperazine salts. The most preferred salts are sodium salts.

Examples of androgens include, without limitation, methyltestosterone; fluoxymesterone; oxandrolone; oxymetholone; stanozolol; 7α-methyl-19-nortestosterone; testosterone; testosterone cypionate; testosterone enanthate; testosterone propionate; danazol; 5α-androstan-3α-ol-16-one; 5α-androstan-3β,16α-diol; 5α-androstan-3β,16α-diol; and 5α-androstan-3β, 17α-diol.

Examples of progestins are set forth in U.S. Pat. No. Re. 36,247 to Plunkett et al., the disclosure of which is incorporated herein in its entirety, and include, but are not limited to, desogestrel; dydrogesterone; ethynodiol diacetate; medroxyprogesterone acetate; levonorgestrel; medroxyprogesterone acetate; hydroxyprogesterone caproate; norethindrone; norethindrone acetate; norethynodrel; allylestrenol; 19-nortestosterone; lynoestrenol; quingestanol acetate; medrogestone; norgestrienone; dimethisterone; ethisterone; cyproterone acetate; chlormadinone acetate; megestrol acetate; norgestimate; norgestrel; desogestrel; trimegestone; gestodene; nomegestrel acetate; progesterone; 5α-pregnan-3β,20α-diol sulfate; 5α-pregnan-3 β,20β-diol sulfate; 5α-pregnan-3β-ol-20-one; 16,5α-pregnen-3′-ol-20-one; and 4-pregnen-20β-ol-3-one-20-sulfate.

Calcium salts may include, without limitation, organic acid salts of calcium such as calcium citrate, calcium lactate, calcium fumurate, calcium acetate, and calcium glycerophosphate, as well as inorganic salts such as calcium chloride, calcium phosphate, calcium sulphate, and calcium nitrate.

Useful dosage forms include without limitation solid oral forms such as tablets, capsules, beads, granules, aggregates, and powders.

A variety of additives can be incorporated into the pharmaceutical compositions of the present invention as will be understood by those skilled in the art. Examples of classes of additives include lubricants, buffering agents, disintegrating agents, stabilizers, foaming agents, pigments, coloring agents, fillers, bulking agents, sweetening agents, flavoring agents, fragrances, release modifiers, adjuvants, plasticizers, flow accelerators, polyols, granulating agents, diluents, binders, buffers, absorbents, glidants, adhesives, antiadherents, acidulants, softeners, resins, demulcents, solvents, surfactants, emulsifiers, elastomers and mixtures thereof.

The present invention will now be described with reference to the following example. It should be appreciated that this example is for the purposes of illustrating aspects of the present invention, and does not limit the scope of the invention as defined by the claims.

### EXAMPLE

Two batches of tablets (Batch 1 and Batch 2) containing hydroxypropylmethyl cellulose (HPMC) of different particle sizes were provided. With the exception of the HPMC particle size in Batch 1 tablets differing from that in Batch 2 tablets, Batch 1 and Batch 2 tablets contained the same active and inactive ingredients in the same percentages as shown below in Table 1, in which all percentages are weight percent as a percent of the total tablet weight:

TABLE 1

| | Batch 1 | Batch 2 |
|---|---|---|
| Ingredient | Tablets | Tablets |
| Mixture of Conjugated Estrogens | 9.26% | 9.26% |
| Lactose | 59.24% | 59.24% |
| Colloidal silicon dioxide | 0.5% | 0.5% |
| HPMC (Batch 1) | 30% | — |
| HPMC (Batch 2) | — | 30% |
| Magnesium stearate | 1% | 1% |

Each batch of tablets was produced in exactly the same manner as follows: The components are charged into a blender and mixed dry for 5-20 minutes. The blend is discharged and compressed into the tablets. The tablets are then coated with a coating material comprising 42.67 weight percent ethylcellulose aqueous suspension, 1.33 Opadry™ color coating material available from Dow weight percent and 56.00% purified water. The tablets formed having the formulations shown in Table 1 were each 180 mg tablets having 0.625 mg dosages of conjugated estrogens.

The HPMC (Batch 1) and HPMC (Batch 2) had the physical properties given in Table 2 below:

TABLE 2

Physical Characteristic

| | Cumulative | HPMC | HPMC |
|---|---|---|---|
| | Weight Percent | (Batch 1) | (Batch 2) |
| | Less Than | Indicated | Indicated |
| | Indicated Size | Size (μm) | Size (μm) |
| Particle | 10% | 17.36 | 9.52 |
| Size | 50% | 88.46 | 59.85 |
| Distribution | 90% | 193.44 | 125.61 |

| | | |
|---|---|---|
| Specific Surface Area (m²/g) | 0.2012 | 0.2677 |
| Viscosity (cps) | 3641 | 4310 |

The differences in viscosities for HPMC (Batch 1) and HPMC (Batch 2) were considered to insignificant differences in terms of viscosity.

The release profiles of the two batches were determined by an HPLC method. The HPLC method used to monitor the amount of bio-active released is based on UV analysis scanned between 190 and 365 nm using a reverse phase system to separate and quantify components of analytical interest.

The release profiles of the three batches are shown in Table 3 below:

TABLE 3

| Time since | Percent Released | Percent Released |
|---|---|---|
| administration | from Batch 1 | from Batch 2 |
| 2 hours | 64% | 40% |
| 5 hours | 92% | 79% |
| 8 hours | 97% | 95% |

The present invention has been described herein with reference to its preferred embodiments. These embodiments do not serve to limit the invention, but are set forth for illustrative purposes. The scope of the invention is defined by the claims that follow.

That which is claimed is:

1. A solid pharmaceutical composition comprising a core, said core comprising:
a bio-active; and
hydroxypropylmethyl cellulose (HPMC) having a particle size distribution wherein the particle size at the 50% cumulative weight percent is less than 75 μm.

2. The composition according to claim 1, wherein the particle size of the HPMC at the 10% cumulative weight percentage is between about 5 μm and 75 μm.

3. The composition according to claim 1, wherein the particle size of the HPMC at the 90% cumulative weight percentage is between about 95 μm and 155 μm.

4. A solid pharmaceutical composition comprising a core, said core comprising:
a bio-active; and
hydroxypropylmethyl cellulose (HPMC) having a particle size distribution wherein the particle size at the 50% cumulative weight percent is less than about 85 μm.

5. The composition according to claim 4, wherein the particle size of the HPMC at the 10% cumulative weight percentage is between about 5 μm and 85 μm.

6. The composition according to claim 4, wherein the particle size of the HPMC at the 90% cumulative weight percentage is between about 75 μm and 175 μm.

7. A solid pharmaceutical composition comprising a core, said core comprising:
a bio-active; and
hydroxypropylmethyl cellulose (HPMC) having a particle size distribution wherein the particle size at the 50% cumulative weight percent is less than about 95 μm.

8. The composition according to claim 7, wherein the particle size of the HPMC at the 10% cumulative weight percentage is between about 5 μm and 95 μm.

9. The composition according to any of claim 7, wherein the particle size of the HPMC at the 90% cumulative weight percentage is between about 85 μm and 205 μm.
